(19) United States
(12) Patent Application Publication
(10) Pub. No.: US 2001/0047036 A1
(43) Pub. Date: Nov. 29, 2001

(54) Composition for improving the proliferative response during adaptation of the gastrointestinal tract and use in short bowel syndrome

(76) Inventors: Jon A. Vanderhoof, Omaha, NE (US); Kathryn A. Bauerly, Davis, CA (US); Eric L. Lien, Malvern, PA (US); John C. Wallingford, Gladwyne, PA (US)

Correspondence Address: Egon E. Berg, American Home Products Corporation, Patent Law Department - 2B, One Campus Drive, Parsippany, NJ, 07054, US

(21) Appl. No.: 09/739,024

(22) Filed: Dec. 14, 2000

Related U.S. Application Data
(60) Provisional application No. 60/219,349, filed on Dec. 17, 1999.

(51) Int. Cl. A61K 31/202

(52) U.S. Cl. 514/560

(57) ABSTRACT

Methods for the treatment of short bowel syndrome in patients in need thereof are provided, comprising the administration to the patients of an effective amount of a formulation comprising arachidonic acid and docosahexanoic acid.

### CROSS REFERENCE TO RELATED APPLICATIONS

This application claims the benefit of U.S. Provisional Application (not yet known), which was converted from U.S. application Ser. No. 09/466,751, filed Dec. 17, 1999.

### FIELD OF THE INVENTION

The present invention is directed to methods of treating patients with short bowel syndrome and to formulations containing long chain polyunsaturated fatty acids (PUFA's) useful in such methods.

### BACKGROUND OF THE INVENTION

Short bowel syndrome is the term used to describe the state of nutrient malabsorption following extensive surgical resection of small intestine (Vanderhoof, J. A., Gastroenterology 113(5): 1767-78 (1997)). The extent to which malabsorption occurs depends not only on the quantitative amount of absorptive tissue removed from the small intestine, but also in the particular portion of the small intestine that is removed. For example, removal of the distal portion of the ileum can result in a more adverse result than removed of the proximal bowel, because removal of the ileocecal valve allows freer communication between the remaining small intestine and the large intestine. The loss of distinct microenvironments impairs the distinct absorption characteristics of the two regions, although there does not appear to be a significant effect on the lower GI microfloral capability to ferment complex carbohydrates (Nordgarachidonic acidrd, I. H. et al., Scandinavian Journal of Gastroenterology 30(9): 897-904 (1995)).

Following the surgical resection of the small intestine, the remaining tissue undergoes an immediate proliferative response of absorptive surface area. The same factors that are associated with malabsorption are associated with the extent of the proliferative response following resection, namely, the extent and location of tissue removed. However, the proliferative response is also influenced by the provision of luminal nutrition.

A number of nutrient substances have been evaluated in an attempt to maximize the proliferative response following resection of the small intestine. Diets high in growth hormone, glutamine, and high carbohydrate-low fat diets have all been studied (see e.g., Byrne, T. P., et al., Annals of Surgery 222(3):254-5 (1995); Scolapio, J. S. et al., Gastroenterology 113(4):1402-5 (1997); Sax, H., Journal of Parenteral and Enteral Nutrition 26(2):123-8 (1998)). Formulas containing amino acids have been studied in an attempt to avoid intact protein irritability and digestion (Bines, J. F. et al., Journal of Pediatric Gastroenterology & Nutrition 26(2):123-8 (1998)). Dietary restrictions of insoluble fiber, oxalates, and lactose have also been proposed (Lykins, T. S. et al., Journal of the American Dietetic Association 98(3):309-15 (1998) are despite evidence that small amounts of lactose are tolerated (Marteau, P. M. et al., Nutrition 13(1):13-16 (1997)).

Fat digestion and absorption is especially problematic when short bowel syndrome occurs in infancy, as fat digestive capability is developmentally limited in early life (e.g., Heineman, E. D. et al., Journal of Pediatric Surgery 31(4):520-5 (1996)). The use of medium chain triglycerides has been suggested as a means to bypass the need to package fat into triglyceride in the intestinal epithelial cell and ease the stress on fat absorption (Goulet, O., European Journal of Medical Research 2(2):79-83 (1997)). At the same time, the provision of essential fatty acids to the patient with short bowel syndrome is an important nutritional objective.

It has been postulated that certain fatty acids may enhance hyperplasia in the remaining GI tract. For example, Kollman, K. A. et al., Journal of Pediatric Gastroenterol Nutrition 28:41-5 (1999) systematically varied the sources of fats and oils used to comprise four diets each of which was fixed in providing 30% of calories from fat. Each diet provided 10% of the fat as soy oil, sufficient to satisfy essential fatty acid requirements. The balance of fat was from hydrogenated coconut oil and docosahexaenoic acid, a 22 carbon long chain polyunsaturated fatty acid of the n-3 class, and arachidonic acid, a 20 carbon long chain fatty acid of the n-6 class. One treatment group had 0% of arachidonic acid and docosahexaenoic acid; one had 5% arachidonic acid and 3.3% docosahexaenoic acid; another group had 15% arachidonic acid and 10% docosahexaenoic acid, and a fourth group had 45% arachidonic acid and 30% docosahexaenoic acid. In this experiment each animal has 80% or the bowel removed. In an additional experiment using two of the diets (no arachidonic acid or docosahexaenoic acid but high safflower oil and 45% arachidonic acid, 30% docosahexaenoic acid) the extent of resection was studied. The extent of resection was 60%, 70% or 80%. In the 80% resected animals, there was a diet dependent response in proliferation, as the very high arachidonic acid/docosahexaenoic acid level resulted in less proliferation in of remaining duodenum DNA than was seen when no arachidonic acid/docosahexaenoic acid were fed. Measures of mucosal mass, protein and sucrase activity did not vary in the duodenum. In marked contrast, the high arachidonic acid/docosahexaenoic acid diet resulted in significantly greater mucosal mass and protein in the ileum than the diet with no arachidonic acid/docosahexaenoic acid. There was also more DNA evident with the high arachidonic acid/docosahexaenoic acid diet, although the difference did not reach statistical significance. These results indicate that there may be particular benefits to a high arachidonic acid/docosahexaenoic acid diet.

When the extent of resection was studied in animals fed one of the two extreme diets, there was no difference in any measure of response among the animals fed safflower oil but resected to different extents. There was, however, a graded response in duodenal DNA amount and sucrase activity, and in ileal mucosal mass, DNA amount and protein amount in the animals fed high arachidonic acid/docosahexaenoic acid diets. In each case the proliferative response was greatest among the most severely resected animals.

These results indicate that not only is the proliferation of remaining intestine augmented by arachidonic acid plus docosahexaenoic acid to a greater extent than when safflower oil is fed, and that there is significantly enhanced mucosal mass when rats are fed high amounts of PUFAs compared to comparable resected rats fed diets containing 10-% soy oil or less PUFAs (3.3% docosahexaenoic acid and 5% arachidonic acid), but that arachidonic acid/docosahexaenoic ACID supplementation is of greatest potential utility when the extent of bowel resection is the greatest.

### SUMMARY OF THE INVENTION

The present invention is directed to methods for the treatment of short bowel syndrome in patients in need thereof comprising administering to the patient an effective amount of a formulation comprising arachidonic acid and docosahexanoic acid. The invention is further directed to formulations suitable for use in such methods.

### DETAILED DESCRIPTION OF THE INVENTION

The present invention is directed to methods of treating short bowel syndrome by administrating to a patent with short bowel syndrome a formulation comprising arachidonic acid and docosahexanoic acid. In the present formulations, the amount of arachidonic acid may be at least equal to or greater than the amount of docosahexanoic acid. Preferably, the amount of arachidonic acid to docosahexanoic acid ranges from 2:1 to 1:1,and more preferably is in the ratio of 1.5:1. The beverages of the invention preferably contain docosahexanoic acid in a quantity by weight of at least 1.5%, more preferably at least 2.2%, and advantageously at least 3.32%, calculated on the total fatty acid content of the beverage. The quantity is advantageously 12%, calculated on the total fatty acid content of the beverage. The beverages of the invention preferably contain arachidonic acid in a quantity by weight of at least 1.5%, more preferably at least 3.32%, and advantageously at least 4.44%, calculated on the total fatty acid content of the beverage. The quantity is advantageously 18%, calculated on the total fatty acid content of the beverage.

The present formulations have been found to be particularly useful in the treatment of short bowel syndrome. In formulations for the treatment of infants with short bowel syndrome, the ratios of arachidonic acid to docosahexanoic acid set forth above also apply.

The present invention is not limited to a particular formulation, as long as the appropriate amounts of arachidonic acid and docosahexanoic acid are contained therein. However, in its preferred form, the present formulation is a complete nutritional beverage comprised of protein, carbohydrate, vitamins and minerals, and containing a specific blend of vegetable fats suitable to achieve a special fatty acid pattern. The formulations of the present invention may be formulated in a liquid form or as a powder intended to be reconstituted in suitable amounts of water prior to consumption. In this embodiment, the invention is formulated in a manner that it is capable of providing the complete nutritional needs of an infant with short bowel syndrome. Both infancy and short bowel syndrome independently place rigorous dietary requirements on the individual, and hitherto, as noted above, there has been no adequate nutritional intervention that simultaneously meets the needs of both infancy and short bowel syndrome.

The fatty acids useful in the present formulations, arachidonic acid and docosahexanoic acid, may be preferably produced in the form of single cell oils. The level of docosahexaenoic acid in the present formula may be equal to or greater than the level of docosahexaenoic acid that has been affirmed by the U.S. Food and Drug Administration as generally recognized as safe for the general population (a total intake of 3 g/day). (21 CFR 184.XX). However, since the intended use of the present formula is in the treatment of short bowel syndrome, the limitations relevant for the general food supply are not relevant as a safety concern. The treatment of short bowel syndrome demands that the particular benefits of the formula, including weight gain, more rapid progression to complete enteral nutrition and reduced occurrences of lactic acidosis, be considered with respect to risks of not using the formula, as well as to any theoretical concerns about physiologically active fatty acids.

As noted above, a preferred embodiment of the present invention is a nutritionally complete infant formula suitable for use in the present method to treat short bowel syndrome in infants and children. Such formulas comprise proteins, carbohydrates, lipids and effective amounts of arachidonic acid and docosahexaenoic acid according to the present invention.

The term “infant formula” will be readily recognizable to those skilled in the art. When diluted or reconstituted, if initially in concentrated or powder form, to the ready to feed state, a typical infant formula will comprise about 60-110 grams of carbohydrates per liter, 10-35 grams of protein per liter, and 20-50 grams of lipid per liter, as well as vitamins, minerals, fibers, emulsifiers, etc. To such an infant formula one can add appropriate amounts of arachidonic acid and docosahexaenoic acid in accordance with the present invention.

Examples of suitable commercially available infant formulas to which the arachidonic acid and docosahexaenoic acid may be added include the S-26, S-26LBW and SMA infant formulas available from Wyeth Nutritionals International Inc.

Preferably, the formulas useful in the present invention do not contain lactose as a carbohydrate, as is typically the case in standard infant formulas, but rather contain maltodextrin. Maltodextrin may be used in conjunction with an alternate form of polymeric glucose, including starches, that have previously been used in infant formula, e.g. tapioca starch. Furthermore, a portion of the carbohydrate, as much as 20%, may be in the form of sucrose to improve the taste of the formulation. This use of carbohydrates allow the formula to be consumed orally for a longer duration of time following resection. However, amount of carbohydrate in order to avoid the excess metabolic production of D-lactic acid contained in the final formulation must be carefully considered by intestinal bacteria.

The present invention will now be illustrated with reference to the following specific examples.

### EXAMPLES

An example of an infant formula formulation suitable for use in the present invention is set forth below:

Formula A

| Ingredients | Grams | % |
|---|---|---|
| Water | 48.5 | 82.5 |
| maltodextrin | 7.1 | 10.6 |
| sodium and calcium caseinates | 1.6 | 2.4 |
| whey protein concentrate | 2.6 | 3.9 |
| vegetable oils | 6.0 | 9.0 |
| minerals | 0.25 | 0.37 |
| vitamins | 0.02 | 0.03 |
| | 66.0 | 100 |

The caloric distribution of the formula A is approximately 28.4% CHO; 16.8% protein, and 54.8% fat; 150 kcal/100 cc.

Various fat blends that may be used to optimize the provision of the high amounts of arachidonic acid and docosahexaenoic acid necessary in the present formulations are shown below. Fat Blends 1-3 contain varying concentrations of arachidonic acid and docosahexaenoic acid in formulas that would comply with FDA's GRAS affirmation on docosahexaenoic acid consumption. These particular fat blends could be best employed in management of short bowel syndrome following the period of rapid proliferation, in order to maintain individuals on high arachidonic acid/docosahexaenoic acid diets.

Fat Blend #1

| | | |
|---|---|---|
| vegetable oil | 92.5% | |
| DHASCO (40% docosahexaenoic acid) | 3.7 | (1.5% docosaliexaenoic acid) |
| ARASCO (40% arachidonic acid) | 3.8 | (1.5% arachidonic acid) |
| | 100 | |

This concentration of docosahexaenoic acid + arachidonic acid, in a ratio of 1:1 will
provide 3.0 g/d LCPs at 1666 kcal/d.

Fat Blend #2

| | |
|---|---|
| vegetable | 83.4% |
| DRASCO (40% docosahexaenoic acid) | 8.3% |
| ARASCO (40% arachidonic acid) | 8.3% |
| | 100 |

This level of DHASCO and ARASCO, in a ratio of 1:1, will provide 3.0 g/d LCP at
750 kcal/d

Fat Blend #3

| | |
|---|---|
| vegetable | 83.4% |
| DHASCO O (40% docosahexaenoic acid) | 5.5% |
| ARASCO (40% arachidonic acid) | 11.1% |
| | 100 |

This level of DHASCO and ARASCO will provide 3.0 g/d LCP at 750 kcal/d, but in
a 2:1 ratio of arachidonic acid to docosahexaenoic acid.

However, the preferred formulation for short bowel syndrome uses larger amounts of arachidonic acid and docosahexaenoic acid than this. In the preferred form of the invention, the level of arachidonic acid is 18% of the fatty acids, and the level of docosahexaenoic acid is 12% of fatty acids (Table 7).

Fat Blend #4

| | |
|---|---|
| vegetable | 10% |
| hydrogenated coconut oil | 15% |
| DHASCO (40% docosahexaenoic acid) | 30% |
| ARASCO (40% arachidonic acid) | 45% |
| | 100 |

The complete fatty acid composition of Fat Blend #4 is set forth below:

Fatty acid distribution on preferred fat blend

| | Fatty acid | % |
|---|---|---|
| Saturates | 8 | 1.5 |
| | 10 | 1.3 |
| | 12 | 8.2 |
| | 14 | 6.7 |
| | 16 | 7.9 |
| | 18 | 5.4 |
| | 20 | 0.4 |
| | 22 | 0.9 |
| | 24 | 0.7 |
| Unsaturates | 18:1w9 | 25.2 |
| | 18:1w7 | 0.3 |
| | 18:2w6 | 8.4 |
| | 18:3w6 | 0.9 |
| | 18:3w3 | 0.9 |
| | 20:1w9 | 0.2 |
| | 20:2w6 | 0.3 |
| | 20:3w6 | 0.6 |
| | 20:4w6 | 18.01 |
| | 20:5w3 | 0.1 |
| | 22:6w3 | 10.89 |

Studies on the mechanism by which diets high in arachidonic acid and docosahexaenoic acid improve the proliferative response indicate that prostaglandin formation is important. Arachidonic acid metabolism may be investigated by the use of pharmacologic agents that selectively block routes of arachidonic acid metabolism by inhibition of cyclooxygenase and lipoxygenase. In rats who have been resected, and who were fed diets in which the fat was provided mainly as arachidonic acid (45% of fatty acids) and docosahexaenoic acid (30% of fatty acids), the proliferative response of the duodenum was increased by treatment with an inhibitor of lipoxygenase. The result of blocking this route of arachidonic acid metabolism is to increase the formation of prostaglandin products via the cyclooxygenase pathway. In the duodenum, mucosal mass, DNA, and protein content were each increased when compared to the resected control rats. In contrast, when cyclooxygenase was inhibited (the pathway that produces prostaglandins, such as thromboxane A₂) there was no change in the mucosal mass, DNA, protein or sucrase activity compared to the resected control rats (Table 1).

TABLE 1
Mucosal mass, DNA, protein and sucrase activity in the duodenum of the
rat Treatment

| | Control | -Lipoxygenase | -Cyclooxygenase |
|---|---|---|---|
| Mucosal mass | 108.0 + 2.5 | 125 + 3.6 | 107.0 + 4.4 |
| (mg/cm) | | | |
| DNA | 287.1 + 5.6 | 323.2 + 10.6 | 276.6 + 11.2 |
| (meg/cm) | | | |
| Protein | 87.6 + 2.0 | 101.3 + 3.1 | 81.0 + 3.0 |
| (mg/cm) | | | |
| Sucrase | 596.4 + 20.7 | 648.0 + 48.6 | 513.9 + 46.2 |
| (umol/cm min) | | | |

Some similar results were observed in the ileum, where mucosal mass and protein content were actually reduced to a statistically significant extent by the cyclooxygenase inhibitor (Table 2).

TABLE 2
Mucosal mass, DNA, protein and sucrase activity in the ileum of the
rat Treatment

| | Control | -Lipoxygenase | -Cyclooxygenase |
|---|---|---|---|
| Mucosal mass | 95.9 + 3.6 | 105.0 + 4.2 | 73.9 + 4.4 |
| (mg/cm) | | | |
| DNA | 252.0 + 9.7 | 257.1 + 15.7 | 223.8 + 16.2 |
| (meg/cm) | | | |
| Protein | 65.9 + 2.1 | 69.5 + 3.3 | 55.4 + 3.5 |
| (mg/cm) | | | |
| Sucrase | 196.4 + 15.6 | 137.4 + 16.7 | 146.7 + 18.7 |
| (umol/cm min) | | | |

A study completed by the present inventors involving the dietary management of a series of children with short bowel syndrome found that a high fat, low carbohydrate, high calorie enteral diet allowed more rapid weaning from total parenteral nutrition, i.e. faster attainment of full enteral feeds, and less occurrence of bacterial overgrowth. Included in the study group were children who previously had difficulty tolerating standard enteral feeds comprised of amino acid or hydrolyzed protein formulas, and who demonstrated improved weight gain after changing to the high fat formula.

These data demonstrate that there is a biochemical linkage from the experimental observations in rats, to human short bowel syndrome. In fact, improved weight gain and reduced occurrence of lactic acidosis in the five individuals who were fed a high fat diet without AA or DHA, may be explained by the learnings from the rat mechanism studies. If the proliferative response is dependent on prostaglandin formation via the cyclooxygenase pathway, then it is possible that the clinical results were entirely dependent on the opportune use of a high fat formulation that contained a high level of the arachidonic acid precursor, linoleic acid. The fat source used in the clinical response was substantially corn oil, which has a preponderance of linoleic acid.

Still, the formation of arachidonic acid from its precursor linoleic acid is rather inefficient compared to the provision of dietary arachidonic acid, and there has been no direct measure of the formation of gastrointestinal prostaglandins formed after feeding corn oil. The dependence of proliferation of the intestine on prostaglandin production, shown in the rat data above, indicates that the arachidonic acid rich diets of this invention are quantitatively superior to any previous dietary treatment for short bowel syndrome.

The present invention may be embodied in other specific forms without departing from the spirit or essential attributes thereof and, accordingly, reference should be made to the appended claims rather than to the foregoing specification as indicating the scope of the invention.

What is claimed is:

1. A method for the treatment of short bowel syndrome in a patient in need thereof, comprising administration to said patient of an effective amount of a formulation comprising arachidonic acid and docosahexaenoic acid.

2. A method as in claim 1, wherein said formulation comprises arachidonic acid and docosahexaenoic acid in a ratio of 2:1 to 1:1.

3. A method as in claim 2, wherein said formulation comprises arachidonic acid and docosahexaenoic acid in a ratio of 1.5:1.

4. A method as in claim 1, wherein said formulation is a nutritionally complete beverage.

5. A method as in claim 4, wherein said nutritionally complete beverage is an infant formula.

6. A nutritionally complete beverage suitable for treating short bowel syndrome comprising arachidonic acid and docosahexaenoic acid.

7. A nutritionally complete beverage as in claim 6, further comprising maltodextrin.

8. A nutritionally complete beverage as in claim 7, which is completely devoid of lactose.

9. A nutritionally complete beverage as in claim 6, wherein the ratio of arachidonic acid to docosahexaenoic acid is 2:1 to 1:1.

10. A nutritionally complete beverage as in claim 9, wherein the ratio of arachidonic acid to docosahexaenoic acid is 1.5:1.

11. A nutritionally complete beverage as in claim 6, which is an infant formula.

12. A method of treating short bowel syndrome in an infant comprising administering to said infant an infant formula comprising arachidonic acid and docosahexaenoic acid.

13. A method as in claim 12, wherein said infant formula comprises arachidonic acid and docosahexaenoic acid in a ratio of 2:1 to 1:1.

14. The method as in claim 13, wherein said infant formula comprises arachidonic acid and docosahexaenoic acid in a ratio of 1.5:1.

15. The method as in claim 12, wherein the provision of high level s of dietary arachidonic acid and docosahaexaenoic acids modify prostaglandin product, increasing the metabolites of the cyclooxygenase pathway that promote proliferation.

16. A nutritionally complete beverage as claimed in claim 6, wherein the docosahexaenoic acid and the arachidonic acid are each present in a quantity by weight of at least 1.5%, calculated on the total fatty acid content of the beverage.

17. A nutritionally complete beverage as claimed in claim 16, containing at least 2.2% by weight of docosahexaenoic acid and at least 3.32% by weight of arachidonic acid, the quantities being calculated on the total fatty acid content of the beverage.

18. A nutritionally complete beverage as claimed in claim 16, containing at least 3.32% by weight of docosahexaenoic acid and at least 4.44% by weight of arachidonic acid, the quantities being calculated on the total fatty acid content of the beverage.

19. A nutritionally complete beverage as claimed in claim 17, containing at least 12% by weight of docosahexaenoic acid and at least 18% by weight of arachidonic acid, the quantities being calculated on the total fatty acid content of the beverage.
